(19) United States
(12) Patent Application Publication
(10) Pub. No.: US 2011/0125233 A1
(43) Pub. Date: May 26, 2011

(54) TISSUE COOLING APPARATUS

(76) Inventors: Ein-Yiao Shen, Taichung (TW); Peng-Chieh Wu, Taichung (TW); Yu-Tzu Huang, Taichung (TW)

(21) Appl. No.: 12/943,550

(22) Filed: Nov. 10, 2010

(30) Foreign Application Priority Data
Nov. 24, 2009 (TW) 98139852

(51) Int. Cl. A61F 7/12

(52) U.S. Cl. 607/105

(57) ABSTRACT

An apparatus for cooling a tissue is provided, which includes a temperature control module, a hypothermic catheter, and a cooling member. The temperature control module includes a regulation unit, a temperature sensor, a processor, a display unit, and a refrigerator. The regulation unit is used to regulate a predetermined temperature. The temperature sensor is used to measure a reference temperature of the tissue. The processor is used to calculate a difference between the predetermined temperature and the reference temperature as a feedback to control the output of a cooling source. The display unit is used to display the temperature information. The refrigerator is used to output the cooling source. The hypothermic catheter includes a primary cooling pipe, to transport the cooling source; and a secondary cooling pipe. The cooling member makes contact with the tissue to reduce its temperature.

### CROSS-REFERENCES TO RELATED APPLICATIONS

This non-provisional application claims priority under 35 U.S.C. §119(a) on Patent Application No. 98139852 filed in Taiwan, R.O.C. on Nov. 11, 2009, the entire contents of which are hereby incorporated by reference.

### BACKGROUND OF THE INVENTION

1. Field of the Invention

The present invention relates to a cooling apparatus, and more particularly, to an apparatus for cooling a tissue.

2. Related Art

With current development in medicine, hypothermia therapy has gradually been widely used in first aid for patients with cerebral hemorrhage or patients with severe trauma. In a hypothermic environment, the body's cell metabolism can be slowed, thus reducing the speed of cell death. A well-known application of this technology is the case of Shao Hsiao-ling, wife of Taichung City Mayor Jason Hu, who was seriously injured in a traffic accident. Shao suffered from an open fracture of the left arm and a severe intracranial hemorrhage, and was in a deep coma, her life hanging by a thread. The medical group, taking the burden of emergency treatment, firstly employed hypothermia therapy to reduce brain and body temperature, so surgery could be completed smoothly, while protecting brain function and reducing the likelihood of multiple complex sequelae.

Hypothermic effect can effectively reduce the oxygen demand for cell survival, particularly for brittle cells in brain tissue and the cardiac muscles of the body. Surgical case histories contain examples of the use of hypothermia technique to protect organs and decrease bleeding volume. In addition, fundamental scientific research has shown that it is possible to control physiological metabolism by means of temperature variation. Currently, fans, cooling blankets, or ice pillows are used to cover the whole body in order to reduce body temperature. However, these measures require considerable human resources, and increase the burden of health care personnel. In addition to the lower efficacy of fans, the other apparatuses sometimes require an impractically large coverage area, resulting in operational difficulties for health care personnel.

Furthermore, conventionally used apparatuses require a person to monitor the temperature at all times, so as to prevent excessively high temperature causing a reduction in therapeutic effect, or a temperature low enough to cause cold related injury. For this reason, some apparatuses use a microcalculater to control water temperature or ice blanket temperature, so as to reduce the inconvenience of human control. However, when an ice pillow experiences water leakage or ice thawing, the application of an electric shock method will be greatly affected. Furthermore, when the body temperature is lower than 33° C., other side effects in addition to cold injury (such as delayed clotting time, reduced platelet number, and delayed wound healing time), may also result. If it is serious, the immune system may be destroyed, and a secondary infection (such as pneumonia), may even be caused.

A catheter is therefore sometimes used to inject a hypothermic saline into the femoral vein, for the purpose of reducing body temperature, or a hydrogel is used inside an energy transfer pad, the water flow and temperature of which are controlled by a machine for the purpose of controlled temperature reduction. Depending on the pathology, the pad is respectively adhered to the abdomen or thigh of a patient, monitored through a calculator, and X ray irradiation or electric shock therapy can be performed at the same time. Thus apparatuses used in hypothermia therapy generally include coverage of ice water or ice, a cooling tent containing a forced gas flow, an external ice blanket, or injection of ice saline.

Accordingly, there is an urgent need to develop a new cooling agent, covering package for reducing temperature externally, or an internal temperature control catheter, providing a better, faster, and safer treatment option, by the present inventors and those skilled in the art.

### SUMMARY OF THE INVENTION

The present invention is directed to provide an apparatus for cooling a tissue, which is used to cool different sites on the body or other tissues, so that the temperature of the tissues is adjusted suitably for a subsequent medical process.

In order to achieve this object, the present invention provides an apparatus for cooling a tissue which includes a temperature control module, a hypothermic catheter, and a cooling member. The temperature control module is used to output a cooling source to the tissue, so that the tissue achieves a predetermined temperature. The temperature control module includes a regulation unit for regulating the predetermined temperature; a temperature sensor, for measuring a reference temperature of the tissue; a processor coupled electrically to the regulation unit and the temperature sensor, to calculate a difference between the predetermined temperature and the reference temperature as a feedback to control the output of the cooling source; a display unit coupled electrically to the regulation unit and the temperature sensor, for displaying the predetermined temperature and the reference temperature; and a refrigerator coupled electrically to the processor, for outputting the cooling source. The hypothermic catheter is connected to the refrigerator, and includes a primary cooling pipe with one end connected to the refrigerator, to transport the cooling source; and a secondary cooling pipe with one end connected to the primary cooling pipe. The cooling member, connected to the secondary cooling pipe, is used in contact with the tissue to reduce its temperature.

The present invention is further directed to provide a tissue cooling apparatus for evaluating a temperature sensing and adjusting system, which is used to cool different sites of the body or other tissues, so that the temperature of the tissues is adjusted suitably for a subsequent medical process.

In order to achieve the above-described object, the present invention provides a tissue cooling apparatus for evaluating a temperature sensing and adjusting system, which includes a temperature control module, a hypothermic catheter, and a cooling member. The temperature control module is used to output a cooling source to the tissue, so that the tissue achieves a predetermined temperature. The temperature control module includes a regulation unit, for regulating the predetermined temperature; a temperature receiver, for receiving a reference temperature of the tissue; a processor coupled electrically to the regulation unit and the temperature receiver, to calculate a difference between the predetermined temperature and the reference temperature as a feedback to control the output of the cooling source; a display unit coupled electrically to the regulation unit and the temperature receiver, for displaying the predetermined temperature and the reference temperature; and a refrigerator coupled electrically to the processor, for outputting the cooling source. The hypothermic catheter is connected to the refrigerator, and includes a primary cooling pipe with one end connected to the refrigerator, to transport the cooling source; and a secondary cooling pipe with one end connected to the primary cooling pipe. The cooling member includes a plurality of cooling needles, connected to the secondary cooling pipe, for contacting with the tissue to reduce its temperature; a temperature probe, for measuring the reference temperature of the tissue; and a wireless transmission device coupled to the temperature probe, for transmitting the reference temperature to the temperature receiver.

The effect of the present invention is that various hypothermic environments suitable for medical requirements can be created and localized temperature reduction can be provided, without the inconvenience of additionally reducing the temperature of tissue in non-medical areas, thus providing advantages to medical operations.

Preferred embodiments of the present invention are described below with reference to the drawings.

### BRIEF DESCRIPTION OF THE DRAWINGS

The present invention will become more fully understood from the detailed description given herein below for illustration only, and thus not limitative of the present invention, wherein:

FIG. 1 is a schematic structural view of a first embodiment of the tissue cooling apparatus according to the present invention;

FIG. 2 is a functional block diagram of the first embodiment of the tissue cooling apparatus according to the present invention;

FIG. 3 is a schematic structural view of a second embodiment of the tissue cooling apparatus according to the present invention; and

FIG. 4 is a functional block diagram of the second embodiment of the tissue cooling apparatus according to the present invention.

### DETAILED DESCRIPTION OF THE INVENTION

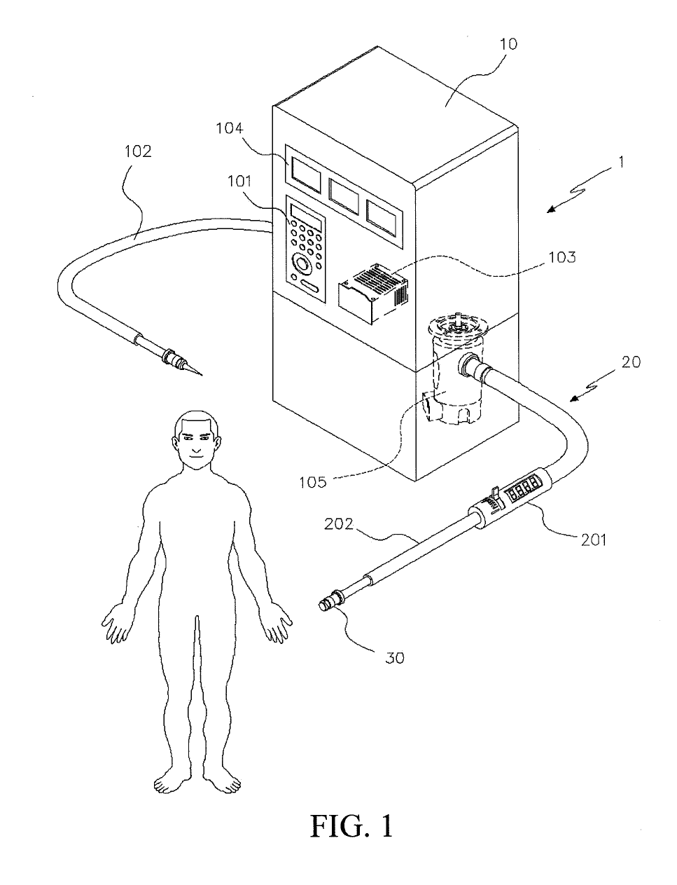

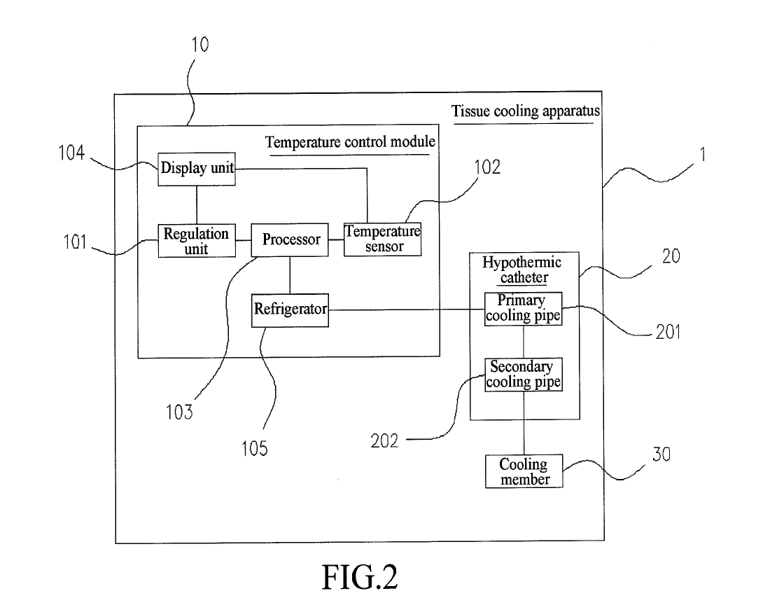

FIG. 1 is a schematic structural view of a first embodiment of the tissue cooling apparatus according to the present invention, and FIG. 2 is a functional block diagram of the first embodiment of the tissue cooling apparatus according to the present invention.

Referring to FIG. 1 and FIG. 2, an apparatus for cooling a tissue 1 is provided in this embodiment, which includes a temperature control module 10, a hypothermic catheter 20, and a cooling member 30. The temperature control module 10 is consisted of multiple components, and is used to output a cooling source to the tissue, so that the tissue achieves a predetermined temperature. The temperature control module 10 includes a regulation unit 101, a temperature sensor 102, a processor 103, a display unit 104, and a refrigerator 105.

The regulation unit 101 is used to regulate the predetermined temperature. Generally, the regulation unit 101 can be configured into an interface device, such as key-press or rotating input, and touch panel, which are commonly used for regulating and setting a predetermined temperature or inputting a specific value.

The temperature sensor 102 is used to measure a reference temperature of the tissue. A contact thermometer can be used as the temperature sensor 102. That is to say, the temperature sensor 102 is in direct contact with a target to be measured, so as to measure the temperature of the target by means of heat conduction. In addition, an infrared thermometer can be used as the measurement tool. Substances at different temperatures will radiate electromagnetic waves with different frequency distributions, and the product of the wavelength λ of the electromagnetic wave with the maximum intensity among the electromagnetic waves with different frequency distributions and temperature T is a constant value, so that the temperature of the measured article can be calculated, for example as in an ear thermometer.

The processor 103 is coupled electrically to the regulation unit 101 and the temperature sensor 102, to calculate a difference between the predetermined temperature and the reference temperature as a feedback to control the output of the cooling source. The processor 103 operates based on the general principle in a calculator.

The display unit 104 is coupled electrically to the regulation unit 101 and the temperature sensor 102, for displaying the predetermined temperature and the reference temperature. The display unit 104 enables operators to observe the temperature reduction, primarily by sight. The display unit can be represented by LED or other display devices, such as an LCD display interface or high-lightness FED device.

The refrigerator 105 is coupled electrically to the processor 103, for outputting the cooling source. When the processor detects that the predetermined temperature is less than the reference temperature, a signal is sent out to inform the refrigerator that the temperature should continue to fall, until the predetermined temperature is equal to the reference temperature. Generally, the devices currently used for reducing temperature include thermoelectric refrigerators and compression refrigerators. One common thermoelectric refrigerator uses a cooling chip for the purpose of reducing temperature. The cooling chip is a semiconductor element, in which an N-type semiconductor material and a P-type semiconductor material are bound into a coupled pair, and energy transfer occurs when the circuit is on. If a junction with a current passing from the N-type element to the P-type element absorbs heat, the junction results in a cold end. On the contrary, if a junction with a current passing from the P-type element to the N-type element releases heat, the junction results in a hot end. A refrigerator device applied herein is achieved on the basis of this principle.

Furthermore, the compression refrigerator is a refrigeration device formed by combining a refrigerant, a compressor, an electromotor, a pipeline and related components, on the basis of the Joule-Thomson principle (as in a refrigerator, for example).

The hypothermic catheter 20 of the tissue cooling apparatus is connected to the refrigerator 105, and includes a primary cooling pipe 201 with one end connected to the refrigerator 105, to transport the cooling source; and a secondary cooling pipe 202 with one end connected to the primary cooling pipe 201. In order to achieve a better effect, a heat pipe (heat catheter) can be used as the structure of the hypothermic catheter 20. In principle, the heat catheter substantially is an enclosed cavity containing a working fluid, in which by means of change in a continuously circulating liquid-gas two-phase flow of the working fluid within the cavity, and convection of the gas-liquid fluid between an endothermic end and an exothermic end, the surface of the cavity exhibits a fast temperature equalizing property in order to achieve heat transfer. The heat pipe of the present invention can thus be an enclosed cavity structure made of a metal material, so as to ensure a fast temperature equalization. The material of the structure can be selected from Cu, Al, or Ti.

In order to reduce errors in transport and the influence from the outside, and to make the control easier and benefits in holding or fixing in operation, the diameter of the primary cooling pipe 201 is larger than that of the secondary cooling pipe 202. When the difference between the predetermined down-regulated temperature and the reference temperature is still too large, the tissue will not receive excessive cooling (even though the primary cooling pipe 201 provides a stronger cooling effect).

A flow meter can be additionally disposed in order to control the gas flow and the liquid flow transported from the primary cooling pipe 201 to the secondary cooling pipe 202.

The cooling member 30 of the tissue cooling apparatus is connected to the secondary cooling pipe 202, for contact with the tissue to reduce its temperature. However, there are various types of tissues, and therefore, depending on the type of the tissue, the cooling member 30 can also be equipped with a needle-like structure, placed vertically into the tissue interior. Alternatively, the cooling member 30 can be equipped with a fixture structure, which provides a cooling source in a clamping manner. It can also be provided with a patch structure or a web-like wrapping structure, which is adhered directly to the tissue.

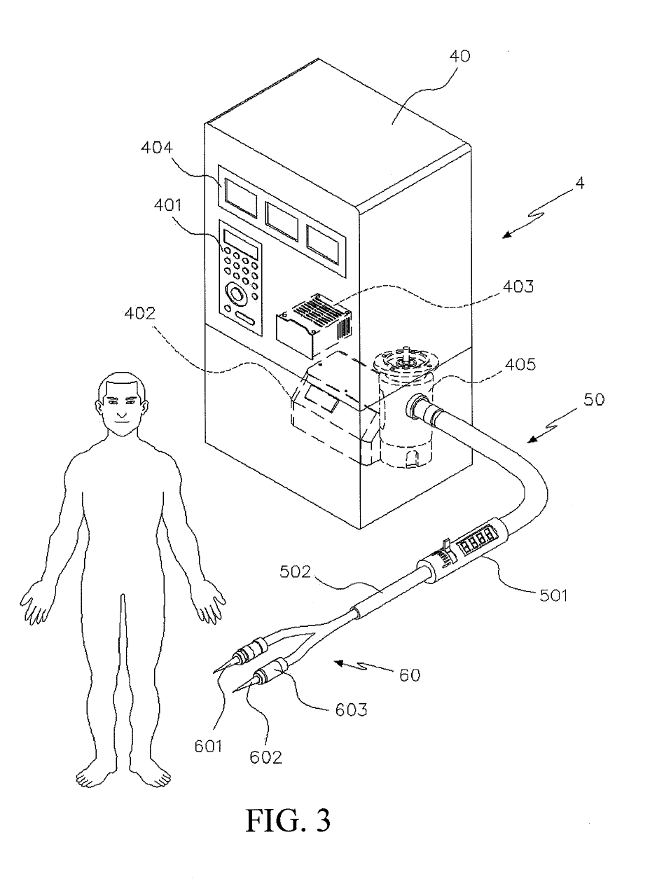

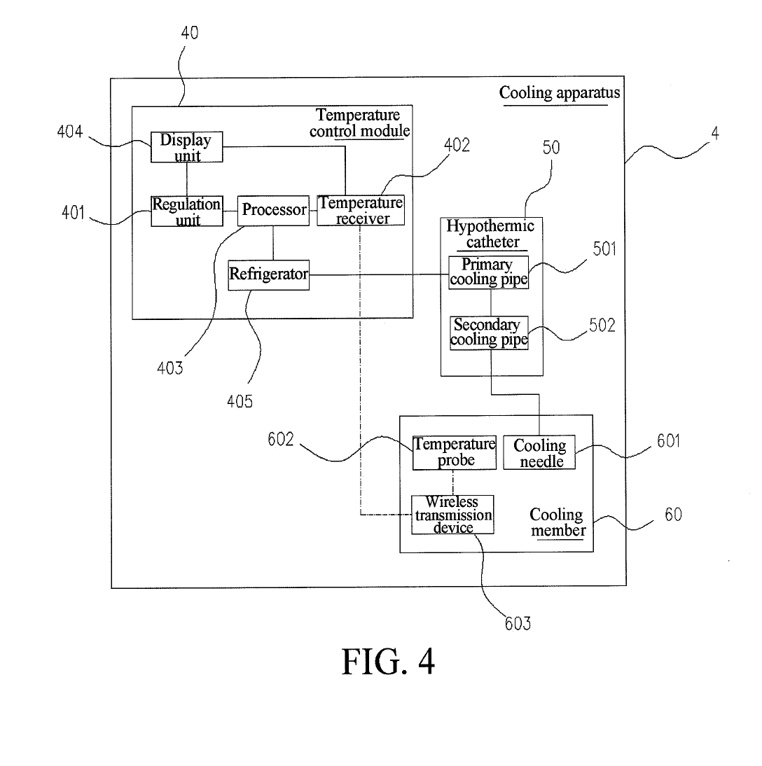

FIG. 3 is a schematic structural view of a second embodiment of the tissue cooling apparatus according to the present invention, and FIG. 4 is a functional block diagram of the second embodiment of the tissue cooling apparatus according to the present invention. Referring to FIGS. 3 and 4, in principle, the structure in this embodiment is the same as that in the first embodiment. According to this embodiment, a tissue cooling apparatus 4 is provided for the evaluation of a temperature sensing and adjusting system, which includes a temperature control module 40, for outputting a cooling source to the tissue, so that the tissue achieves a predetermined temperature; a hypothermic catheter 50; and a cooling member 60. The temperature control module 40 includes a regulation unit 401, for regulating the predetermined temperature; a temperature receiver 402, for receiving a reference temperature of the tissue; a processor 403 coupled electrically to the regulation unit 401 and the temperature receiver 402, to calculate a difference between the predetermined temperature and the reference temperature as a feedback to control the output of the cooling source; a display unit 404 coupled electrically to the regulation unit 401 and the temperature receiver 402, for displaying the predetermined temperature and the reference temperature; and a refrigerator 405 coupled electrically to the processor 403, for outputting the cooling source. The hypothermic catheter 50 is connected to the refrigerator 405 and includes a primary cooling pipe 501 with one end connected to the refrigerator 405, to transport the cooling source; and a secondary cooling pipe 502 with one end connected to the primary cooling pipe 501. The cooling member 60 includes a plurality of cooling needles 601, connected to the secondary cooling pipe 502, for contacting with the tissue to reduce its temperature; a temperature probe 602, for measuring the reference temperature of the tissue; and a wireless transmission device 603 coupled to the temperature probe 602, for transferring the reference temperature to the temperature receiver 402.

The difference between this embodiment and the first embodiment is that the temperature probe 602 disposed on the cooling member 60 is used to measure the reference temperature of the tissue instead, and the temperature sensor of the temperature control module in the first embodiment is replaced by the temperature receiver 402. After measuring the reference temperature at the tissue end by the temperature probe 602, the reference temperature is transferred to the temperature receiver 402 by the wireless transmission device 603. The data is then transmitted to the processor 403, for example, based on the infrared wireless transmission technique or the Bluetooth-based wireless transmission technique, and calculated to determine the cooling source output.

In addition, the cooling member 60 in this embodiment is different from that in the first embodiment. The cooling member 60 in this embodiment includes a plurality of cooling needles 601 which can be a fixed or adjustable structure. When the plurality of cooling needles 601 is configured with an adjustable structure, they can be designed to be horizontally adjustable or vertically adjustable. The cooling needles 601 with a horizontally adjustable structure enable the temperature of the tissue to be reduced in a large range for the purpose of cooling; and the cooling needles 601 with a vertically adjustable structure allow localized temperature reduction to be performed on the tissue at a relatively deep position. Other than the members referred to previously, the remaining means and the principle in this embodiment are the same as those in the first embodiment, and are therefore not described here again.

The effect of the present invention is that various hypothermic environments suitable for medical requirements can be created and localized temperature reduction can be provided, and especially by using horizontal or vertical cooling needles, whether across a large range or in a deep position, temperature reducing of the tissue can be achieved, without the inconvenience of additionally reducing the temperature of tissues in non-medical areas, thus being advantageous to medical operations.

While the present invention has been described by the way of example and in terms of the preferred embodiments, it is to be understood that the invention need not to be limited to the disclosed embodiments. On the contrary, it is intended to cover various modifications and similar arrangements included within the spirit and scope of the appended claims, the scope of which should be accorded the broadest interpretation so as to encompass all such modifications and similar structures.

What is claimed is:

1. An apparatus for cooling a tissue, comprising:
a temperature control module, for outputting a cooling source to the tissue, so that the tissue achieves a predetermined temperature, wherein the temperature control module comprises:
a regulation unit, for regulating the predetermined temperature;
a temperature sensor, for measuring a reference temperature of the tissue;
a processor, coupled electrically to the regulation unit and the temperature sensor, to calculate a difference between the predetermined temperature and the reference temperature as a feedback to control the output of the cooling source;
a display unit, coupled electrically to the regulation unit and the temperature sensor, for displaying the predetermined temperature and the reference temperature; and
a refrigerator, coupled electrically to the processor, for outputting the cooling source;
a hypothermic catheter, connected to the refrigerator, and comprising:
a primary cooling pipe, having one end connected to the refrigerator, to transport the cooling source; and
a secondary cooling pipe, having one end connected to the primary cooling pipe; and
a cooling member, connected to the secondary cooling pipe, for contacting with the tissue to reduce its temperature.

2. The cooling apparatus according to claim 1, wherein the temperature sensor is a contact thermometer.

3. The cooling apparatus according to claim 1, wherein the temperature sensor is an infrared thermometer.

4. The cooling apparatus according to claim 1, wherein the refrigerator is a thermoelectric refrigerator.

5. The cooling apparatus according to claim 4, wherein the thermoelectric refrigerator comprises a cooling chip.

6. The cooling apparatus according to claim 1, wherein the refrigerator is a compression refrigerator.

7. The cooling apparatus according to claim 1, wherein the hypothermic catheter is a heat pipe.

8. The cooling apparatus according to claim 7, wherein the heat pipe is an enclosed cavity structure made of a metal material.

9. The cooling apparatus according to claim 8, wherein the metal material is Cu.

10. The cooling apparatus according to claim 8, wherein the metal material is Al.

11. The cooling apparatus according to claim 8, wherein the metal material is Ti.

12. The cooling apparatus according to claim 1, wherein a diameter of the primary cooling pipe is larger than that of the secondary cooling pipe.

13. The cooling apparatus according to claim 12, further comprising: a flow meter, connected to the primary cooling pipe, for controlling a gas flow and a liquid flow transported from the primary cooling pipe to the secondary cooling pipe.

14. The cooling apparatus according to claim 1, wherein the cooling member is a needle-like structure.

15. The cooling apparatus according to claim 1, wherein the cooling member is a fixture structure.

16. The cooling apparatus according to claim 1, wherein the cooling member is a patch structure.

17. The cooling apparatus according to claim 1, wherein the cooling member is a web-like wrapping structure.

18. A tissue cooling apparatus for evaluating a temperature sensing and adjusting system, comprising:
a temperature control module, for outputting a cooling source to the tissue, so that the tissue achieves a predetermined temperature, wherein the temperature control module comprises:
a regulation unit, for regulating the predetermined temperature;
a temperature receiver, for receiving a reference temperature of the tissue;
a processor, coupled electrically to the regulation unit and the temperature receiver, to calculate a difference between the predetermined temperature and the reference temperature as a feedback to control the output of the cooling source;
a display unit, coupled electrically to the regulation unit and the temperature receiver, for displaying the predetermined temperature and the reference temperature; and
a refrigerator, coupled electrically to the processor, for outputting the cooling source;
a hypothermic catheter, connected to the refrigerator, and comprising:
a primary cooling pipe, having one end connected to the refrigerator, to transport the cooling source; and
a secondary cooling pipe, having one end connected to the primary cooling pipe; and
a cooling member, comprising:
a plurality of cooling needles, connected to the secondary cooling pipe, for contacting with the tissue to reduce its temperature;
a temperature probe, for measuring the reference temperature of the tissue; and
a wireless transmission device, coupled to the temperature probe, for transmitting the reference temperature to the temperature receiver.

19. The cooling apparatus according to claim 18, wherein the cooling needles have a fixed structure.

20. The cooling apparatus according to claim 18, wherein the cooling needles have an adjustable structure.

21. The cooling apparatus according to claim 20, wherein the cooling needles have a horizontally adjustable structure.

22. The cooling apparatus according to claim 20, wherein the cooling needles have a vertically adjustable structure.

23. The cooling apparatus according to claim 18, wherein the wireless transmission device is an infrared transmission device.

24. The cooling apparatus according to claim 18, wherein the wireless transmission device is a Bluetooth-based transmission device.
